SEQUENCE LISTING (14 sequences)

SEQ ID NO: 1 - DNA, 16 bp, Artificial Sequence
  Description of Artificial Sequence Synthetic primer
tccgttgtggatctga

SEQ ID NO: 2 - DNA, 18 bp, Artificial Sequence
  Description of Artificial Sequence Synthetic primer
caccaccttcttgatgtc

SEQ ID NO: 3 - DNA, 22 bp, Artificial Sequence
  Description of Artificial Sequence Synthetic probe
atgccgcctggagaaacctgcc

SEQ ID NO: 4 - DNA, 17 bp, Artificial Sequence
  Description of Artificial Sequence Synthetic primer
cacaacctggtgctctt

SEQ ID NO: 5 - DNA, 18 bp, Artificial Sequence
  Description of Artificial Sequence Synthetic primer
cttcgtgtagcttcttga

SEQ ID NO: 6 - DNA, 18 bp, Artificial Sequence
  Description of Artificial Sequence Synthetic probe
cttcgtgtagcttcttga

SEQ ID NO: 7 - DNA, 16 bp, Artificial Sequence
  Description of Artificial Sequence Synthetic primer
gacacgcttcagtttg

SEQ ID NO: 8 - DNA, 16 bp, Artificial Sequence
  Description of Artificial Sequence Synthetic primer
aagcagcactcttcca

SEQ ID NO: 9 - DNA, 21 bp, Artificial Sequence
  Description of Artificial Sequence Synthetic probe
ctgttcggaccgcggcttcta

SEQ ID NO: 10 - DNA, 16 bp, Artificial Sequence
  Description of Artificial Sequence Synthetic primer
aagcagcactcttcca

SEQ ID NO: 11 - DNA, 16 bp, Artificial Sequence
  Description of Artificial Sequence Synthetic primer
gggtgctaagcagttg

SEQ ID NO: 12 - DNA, 26 bp, Artificial Sequence
  Description of Artificial Sequence Synthetic probe
tgacaacagcctcaagatcatcagca

SEQ ID NO: 13 - DNA, 295 bp, Artificial Sequence
  Description of Artificial Sequence Synthetic polynucleotide
gngncanacatttctctggcctactggccggtacctgagctcgctagcctcgagcggggtgcaggagagaagagactggctgggaggagggagagggcgggagcaaaggcgcgggggagtggtcagcagggagaggggtggggggtagggtggagcccgggctgggaggagtcggctcacacataaaagctgaggcactgaccagcctgcaaactccaagcttggcaatccggtactgttggtaaagccaccatggaagatgccaaaaacattaagaagggcccagcgccattct

SEQ ID NO: 14 - DNA, 924 bp, Artificial Sequence
  Description of Artificial Sequence Synthetic polynucleotide
ctgaattctctagaacgggcattcagcatggccttggcgctctgcggctccctgccccccacccagcctcgcccccgcgcaccccccagcccctgcgaccgccgccccccccccggggccccagggcccccagcccgcaccccccgccccgctcttggctcgggttgcgggggcgggccgggggcggggcgagggctccgcgggcgcccattggcgcgggcgcgaggccagcgaggcccgcgcgggccctgggccgcgggctggcgcgactataagagccgggcgtgggcgcccgcagttcgcctgctctccggcggagctgcgtgaggcccggccggccccggccccccccttccggccgcccccgcctcctggcccacgcctgcccgcgctctgcccaccagcgcctccatcgggcaaggcggccccgcgtcgacgccgcccgctgcctcgctgctgactcccgtcccgggcgccgtccgcggggtcgcgctccgccgggcctgcggattccccgccgcctcctcttcatctacctcaactccccccatccccgcttcgcccgaggaggcggttttcactgccctgtgcagagatgagccgggggtgcaggatgggagcccatggcacttcgctacgggatggtccagggctcccggttgggggtgcaggagagaagagactggctgggaggagggagagggcgggagcaaaggcgcgggggagtggtcagcagggagaggggtggggggtagggtggagcccgggctgggaggagtcggctcacacataaaagctgaggcactgaccagcctgcaaactggacattagcttctcctgtgaaagagacttccagcttcctcctcctcctcttcctcctcctcctcctgccccagcgagccttctgctgagctgtaggtaacc

(19) United States
(12) Patent Application Publication
(10) Pub. No.: US 2013/0274235 A1
(43) Pub. Date: Oct. 17, 2013

(54) TREATMENT OF MOTOR NEURON DISEASE

(71) Applicants: Ole Isacson, Cambridge, MA (US); Anna Charlotta Teresia Magnuson Osborn, Belmont, MA (US)

(72) Inventors: Ole Isacson, Cambridge, MA (US); Anna Charlotta Teresia Magnuson Osborn, Belmont, MA (US)

(73) Assignee: THE GENERAL HOSPITAL CORPORATION, Belmont, MA (US)

(21) Appl. No.: 13/876,177

(22) PCT Filed: Oct. 10, 2011
(86) PCT No.: PCT/US2011/055645

§ 371 Date: May 7, 2013

Related U.S. Application Data
(60) Provisional application No. 61/391,539, filed on Oct. 8, 2010.

(51) Int. Cl. A61K 31/53; A61K 31/431; A61K 31/573; A61K 31/554; A61K 31/4178; A61K 31/522; A61K 31/155; A61K 31/553

(52) U.S. Cl. CPC A61K 31/53; A61K 31/155; A61K 31/431; A61K 31/553; A61K 31/554; A61K 31/4178; A61K 31/522; A61K 31/573
USPC 514/180; 514/634; 514/243; 514/196; 514/211.13; 514/211.07; 514/382; 514/263.38; 506/10

(57) ABSTRACT

Provided herein are methods and compositions for the treatment of motor neuron diseases including, for example, amyotrophic lateral sclerosis. Suitable therapeutic agents include, for example, agents that up-regulate the expression IGF-II or guanine deaminase in a cell.

### CROSS REFERENCE TO RELATED APPLICATIONS

This application claims priority from U.S. Provisional Patent Application No. 61/391,539, filed Oct. 8, 2010, herein incorporated by reference in its entirety.

### STATEMENT OF GOVERNMENT-SPONSORED RESEARCH

This invention was made with United States government support awarded by the following agency: ALS Research Program Therapeutic Development Award/DOD USAMRAA W81XWH-08-1-0496. The United States government has certain rights in the invention.

### FIELD OF THE INVENTION

The present invention relates to compositions and methods for treating motor neuron diseases.

### BACKGROUND OF THE INVENTION

The following discussion of the background of the invention is merely provided to aid the reader in understanding the invention and is not admitted to describe or constitute prior art to the present invention.

Neurodegenerative diseases are characterized by the selective vulnerability of specific neuronal populations to toxic processes of genetic and/or environmental origin. Somatic motor neurons degenerate in diseases such as amyotrophic lateral sclerosis (ALS), spinal muscular atrophy (SMA), and spinobulbar muscular atrophy (SBMA). However, not all somatic motor neurons are equally affected by the events leading to degeneration. While ventral spinal motor neurons are affected in all three diseases, and motor neurons of the lower cranial nerves (e.g. hypoglossal (CN12)) degenerate in ALS and SBMA, upper cranial nerves (e.g. oculomotor/trochlear (CN3/4)) are generally spared in SMA, ALS, and SBMA. While SMA is recessively inherited and caused by loss of functional survival of motor neuron (SMN1) protein, SBMA is an X-linked disorder, caused by the expansion of CAG repeats in the androgen receptor gene. The vast majority of ALS cases appear sporadic (sALS) (˜90%). However, ALS can be inherited dominantly (fALS) (˜10%) due to mutations in superoxide dismutase 1 (SOD1), angiogenin or the DNA/RNA-binding proteins TDP-43 or FUS and recessively due to FUS mutations. Furthermore, while homozygous deletion of SMN1 is not associated with ALS, abnormal SMN1 copy number appears to increase the risk of ALS. Importantly, the pathology and pattern of selective motor neuron vulnerability is similar in fALS and sALS, indicating that differential vulnerability among motor neurons is independent of the cause of disease.

Neurodegeneration in many diseases appears to involve cell-autonomous and non-cell-autonomous events. fALS model data indicate that non-cell-autonomous events are instrumental for disease progression, while factors intrinsic of motor neurons are crucial for initiation of degeneration. Furthermore, neuronal death in Huntington's disease, a polyglutamine expansion disease like SBMA, involves intrinsic and exogenous events, suggesting that SBMA could be due to a combination of these two.

### SUMMARY OF THE INVENTION

The present inventions are based on the discovery that increasing the expression of IGF-II and/or guanine deaminase is useful for treating or preventing motor neuron disease.

In one aspect, the invention provides a method for treating motor neuron disease in a patient (in some embodiments, a human patient) by administering a therapeutic agent that up-regulates IGF-II preferably at least 2-fold. In some embodiments, IGF-II may be up-regulated between 2-fold and 10-fold.

In some embodiments, the therapeutic agents are selected from the group consisting of: Agomelatine, Aliskiren, Amlodipine, Amoxapine, Aranidipine, Aspartame, Atomoxetine HCl, Azelnidipine, Azelastine HCl, Barnidipine, Benidipine, Bumetanide, Carprofen, Carvedilol, Ceftibuten, Ceftriaxone, Cefixime, Ceftazidime, Ceftriaxone, Cefcapene, Cefdaloxime, Cefdinir, Cefditoren, Cefetamet, Cefinenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, Cetirizine HCl, Chlorpromazine, Cilnidipine, Citalopram, Clevidipine, Dapoxetine, Deracoxib, Dexamethasone acetate, Dicloxacillin sodium, Diclofenac, Diclofenac sodium, Diflunisal, Diltiazen, Diltiazem HCl, Droxicam, Efonidipine, Eletriptan Hydrobromide, Escitalopram, Felodipine, Fenoprofen, Flurbiprofen, Fluoxetine, Fluvoxamine, Fluvoxamine Maleate, Glyburide, Guaifenesin, Guanabenz, Guanabenz Acetate, Hydrazine, Hydralazine HCl, Ibuprofen, Indalpine, Indomethacin, Isoxicam, Ketoprofen, Lacidipine, Lercanidipine, Levocarnitine, Levodopa, Lornoxicam, LY-156735, Manidipine, Maprotiline, Mazindol, Meclofenamic acid, Mefenamic acid, Melatonin, Meloxicam, Meloxicam sodium, Methyldopate, Methyldopate HCl, Mianserin, Mirtazapine, Modafinil, R-modafinil, Niacinamide, Nicardipine, Nicotine ditartrate, Nifedipine, Nilvadipine, Nisoldipine, Nitrendipine, Olanzapine, Olmesartan Medoxomil, Omeprazole, Oxacephem, Oxaprozin, Paroxetine, Pramipexole, Pranidipine, Ramelteon, Riboflavin, Riluzole, Ropinirole, Sertraline, Setiptiline, Simvastatin, Sulindac, Tacrine HCl, Tasimelteon, Tenoxicam, Thiamine, Thiamphenicol, Tolfenamic acid, Valacyclovir HCl, Vardenafil HCl, Valdecoxib, Vitamin B1 (thiamine), Zimelidine, Zolpidem, sildenafil HCl, tadalafil HCl, or esters or pharmaceutically acceptable salts thereof. In preferred embodiments, the therapeutic agent is selected from Vardenafil HCl and guanabenz acetate.

In some embodiments, the dose and form/route of a therapeutic agent may be selected from the table below. The doses listed below represent doses known to be therapeutically effective for the treatment of diseases other than motor neuron disease. For the treatment of motor neuron disease, the dose of any individual therapeutic agent may be 10%, 20%, 30%, 40%, 50%, 75%, 100%, 150%, 200% or more greater than the dosage indicated below. Alternatively, the dose of any individual therapeutic agent may be 10%, 20%, 30%, 40%, 50%, 60%, 70%, 80% or 90% of the dosage indicated below.

| Therapeutic Agent | Dose | Dosage form/Route | Frequency |
|---|---|---|---|
| Eletriptan HBr | 20 mg or 40 mg | Oral tablet | Not more than 80 mg/ |
| | | | day |
| Modafinil | 100-400 mg | Oral tablet | |
| Dicloxacillin sodium | EQ 62.5 mg | Oral capsule or oral suspension | Four times daily |
| | suspension, EQ 125, | | |
| | 250, or 500 mg | | |
| | capsule | | |
| Thiamphenicol | 0.5-3 g | IM, IV, or oral | Once daily |
| Ceftibuten | EQ 400 mg oral, EQ | Oral capsule or oral suspension | Once daily |
| | 90 mg suspension | | |
| Tacrine HCl | EQ 10, 20, 30, or 40 mg | Oral capsule | Four times daily |
| Fluoxetine | 20 mg/5 ml solution, | Oral capsule, solution, or tablet | 1-2 times per day |
| | 60 mg tablet, EQ 10 | | |
| | or 20 mg capsule | | |
| Citalopram | EQ 10 mg/5 ml | Oral tablet or solution | Once per day |
| | solution, EQ 10, 20, | | |
| | 40, or 60 mg tablet | | |
| Fluvoxamine maleate | 25, 50, or 100 mg | Oral tablet | Once per day |
| Amoxapine | 25, 50, or 100 mg | Oral tablet | 2-3 times daily |
| Atomoxetine HCl | 10, 18, 25, 40, 60, | Oral capsule | 1-2 times daily |
| | 80, 100 mg | | |
| Olmesartan medoxomil | 5, 20, or 40 mg | Oral tablet | 1-2 times daily |
| Guanabenz acetate | EQ 4 or 8 mg | Oral tablet | Twice per day |
| Hydralazine HCl | 20 mg/ml injection, | Injection or oral tablet | As needed |
| | 10, 25, 50, or 100 mg | | |
| | oral tablet | | |
| Methyldopate HCl | 20-40 mg/ml, | Injection, oral suspension, | One divided dose |
| | | | at 6-hour intervals |
| Diltiazem HCl | 5 mg/ml injection, | Oral ER capsule, oral tablet, | 5-15 mg/hour over |
| | 30, 60, 90, or 120 mg | injection | 24 hours, injection, |
| | tablet; 120, 180, | | orally 3-4 times |
| | or 240 ER capsule | | daily |
| Glyburide | 1.25, 2.5, or 5 mg | Oral tablet | 1-2 times daily |
| Flurbiprofen | 50 or 100 mg | Oral tablet | 3-4 times daily, not |
| | | | to exceed 300 mg/ |
| | | | day |
| Carprofen | 2 mg/lb | Oral tablet | Once daily |
| Meloxicam sodium | 7.5 or 15 mg | Oral tablet | Once daily |
| Diclofenac potassium | 50 mg | Oral tablet | 2-3 times daily |
| Levodopa | 100, 250, or 500 mg | Oral capsule | 1-3 times daily |
| Olanzapine | 5 mg, 15 mg, 20 mg | Oral tablet | Once daily |
| Chlorpromazine HCl | 10, 25, 50, 100, or | Injection, oral tablet | 3-4 times daily |
| | 200 mg oral tablet, | | orally, injection |
| | 25 mg/ml injection | | from one-time to |
| | | | every 2-4 hours |
| Valacyclovir HCl | EQ 500 mg or 1 g | Oral tablet | 1-2 times daily |
| Levocarnitine | 200 mg/ml injection, | Oral tablet, oral solution, injection | 1-3 times daily |
| | 1 g/10 ml oral | | orally, 50 mg/kg/ |
| | solution, 330 mg | | day |
| | tablet | | injection |
| Ropinirole | .25, .5, 1, 2, 3, 4, or | Oral tablet | 1-3 times daily |
| | 5 mg | | |
| Vardenafil HCl | 2.5, 5, 10, or 20 mg | Oral tablet | As needed, usually |
| | | | once daily |
| Guaifenesin | 600 mg, 1.2 g | Oral ER tablet | 2-6 times daily |
| Omeprazole | 10, 20, or 40 mg | Delayed release capsule or tablet | Once daily |
| Cetirizine HCl | 5 to 10 mg | Oral capsule, tablet, chewable | 1-2 times daily |
| | | tablet, syrup, solution | |
| Azelastine HCl | EQ 0.125 mg spray, | Nasal spray or ophthalmic drops | Twice daily |
| | 0.05% drops | | |
| Ramelteon | 8 mg | Oral tablet | Once daily |
| Nicotine ditartrate | 7-21 mg | transdermal, nasal spray, inhalation | 1-2 times daily |
| Zolpidem | 5 or 10 mg | Oral tablet | Once daily |
| Aspartame | Up to 50 mg/kg | Oral tablet or capsule | Once daily |
| Thiamine pyrophosphate | 0.2-100 mg | IV, IM solution, oral solution, | Once daily |
| | | tablet | |
| Riboflavin | 0.2-120 mg | Oral tablet | 1-2 times daily |
| Niacinamide | 4% cream or 25-500 mg | oral tablet, topical cream | Once daily or as |
| | | | needed |
| Dexamethasone acetate | 0.1% gel, EQ 8 mg/ml | Topical gel, injection | Single dose |
| | injection | | injection, topical |
| | | | gel as needed |
| Nifedipine | 10, 20, 30, 90 mg | Oral capsule | 1-3 times daily |
| Sildenafil Citrate | 20, 50, or 100 mg | Oral tablet | As needed, not to |
| | | | exceed once per |
| | | | day |
| Tadalafil HCl | 2.5, 5, 10, or 20 mg | Oral tablet | As needed, not to |
| | | | exceed once per |
| | | | day |

In some embodiments, the motor neuron disease is selected from the group consisting of: amyotrophic lateral sclerosis (ALS), progressive bulbar palsy, spinobulbar muscular atrophy, pseudobulbar palsy, primary lateral sclerosis, progressive muscular atrophy (PMA), spinal muscular atrophy, and post-polio syndrome.

In some embodiments, the therapeutic agents are administered in an amount and duration sufficient to increase the expression of IGF-II in the motor neurons of the patient. The increased IGF-II expression can be 2-fold, 3-fold, 4-fold, 5-fold, 6-fold, 7-fold, 10-fold, or more.

In another aspect, the invention provides a method of screening a drug for activity against a motor neuron disease comprising the steps of treatment of cells with the drug, measuring the level of IGF-II gene expression, comparing the level of IGF-II gene expression in the treated cells with a control, and determining that the drug has activity against a motor neuron disease if the level of IGF-II has increased at least 2-fold. In some embodiments, the level of IGF-II has increased between 2-fold and 10-fold. In some embodiments, the level of IFG-II gene expression is determined by measuring the level of IGF-II mRNA and/or protein.

In still another aspect, the invention provides a method for treating motor neuron disease in a patient (in some embodiments, a human patient) by administering a therapeutic agent wherein the therapeutic agent up-regulates guanine deaminase at least 2-fold. In some embodiments, guanine deaminase may be up-regulated between 2-fold and 10-fold.

In some embodiments, the therapeutic agents are selected from the group consisting of: Agomelatine, Aliskiren, Amlodipine, Amoxapine, Aranidipine, Aspartame, Atomoxetine HCl, Azelnidipine, Azelastine HCl, Barnidipine, Benidipine, Bumetanide, Carprofen, Carvedilol, Ceftibuten, Ceftriaxone, Cefixime, Ceftazidime, Ceftriaxone, Cefcapene, Cefdaloxime, Cefdinir, Cefditoren, Cefetamet, Cefinenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, Cetirizine HCl, Chlorpromazine, Cilnidipine, Citalopram, Clevidipine, Dapoxetine, Deracoxib, Dexamethasone acetate, Dicloxacillin sodium, Diclofenac, Diclofenac sodium, Diflunisal, Diltiazen, Diltiazem HCl, Droxicam, Efonidipine, Eletriptan Hydrobromide, Escitalopram, Felodipine, Fenoprofen, Flurbiprofen, Fluoxetine, Fluvoxamine, Fluvoxamine Maleate, Glyburide, Guaifenesin, Guanabenz, Guanabenz Acetate, Hydrazine, Hydralazine HCl, Ibuprofen, Indalpine, Indomethacin, Isoxicam, Ketoprofen, Lacidipine, Lercanidipine, Levocarnitine, Levodopa, Lornoxicam, LY-156735, Manidipine, Maprotiline, Mazindol, Meclofenamic acid, Mefenamic acid, Melatonin, Meloxicam, Meloxicam sodium, Methyldopate, Methyldopate HCl, Mianserin, Mirtazapine, Modafinil, R-modafinil, Niacinamide, Nicardipine, Nicotine ditartrate, Nifedipine, Nilvadipine, Nisoldipine, Nitrendipine, Olanzapine, Olmesartan Medoxomil, Omeprazole, Oxacephem, Oxaprozin, Paroxetine, Pramipexole, Pranidipine, Ramelteon, Riboflavin, Riluzole, Ropinirole, Sertraline, Setiptiline, Simvastatin, Sulindac, Tacrine HCl, Tasimelteon, Tenoxicam, Thiamine, Thiamphenicol, Tolfenamic acid, Valacyclovir HCl, Vardenafil HCl, Valdecoxib, Vitamin B1 (thiamine), Zimelidine, Zolpidem, sildenafil HCl, tadalafil HCl, or esters or pharmaceutically acceptable salts thereof. In a preferred embodiment, the therapeutic agent is Vardenafil HCl.

In further embodiments, the motor neuron disease is selected from the group consisting of: amyotrophic lateral sclerosis (ALS), progressive bulbar palsy, spinobulbar muscular atrophy, pseudobulbar palsy, primary lateral sclerosis, progressive muscular atrophy (PMA), spinal muscular atrophy, and post-polio syndrome.

In yet further embodiments, the therapeutic agents are administered in an amount and duration sufficient to increase the expression of guanine deaminase in the motor neurons of the patient. The increased guanine deaminase expression can be 2-fold, 3-fold, 4-fold, 5-fold, 6-fold, 7-fold, 10-fold, or more.

By “treating” is meant the medical management of a patient with the intent that a cure, amelioration, or prevention of a motor neuron disease will result. This term includes active treatment, that is, treatment directed specifically toward improvement of a motor neuron disease, and also includes causal treatment, that is, treatment directed toward removal of the cause of the disease, pathological condition, or disorder. In addition, this term includes palliative treatment, that is, treatment designed for the relief of symptoms rather than the curing of the disease; preventive treatment, that is, treatment directed to prevention of the disease; and supportive treatment, that is, treatment employed to supplement another specific therapy directed toward the improvement of the disease. The term “treating” also includes symptomatic treatment, that is, treatment directed toward constitutional symptoms of the disease.

By “an amount sufficient” is meant the amount of a compound, alone or in combination with another therapeutic regimen, required to treat, prevent, or reduce a metabolic disorder such as diabetes in a clinically relevant manner. A sufficient amount of an active compound used to practice the present invention for therapeutic treatment of conditions caused by or contributing to motor neuron disease varies depending upon the manner of administration, the age, body weight, and general health of the patient.

As used herein, “motor neuron disease” or MND refers to a group of progressive neurological disorders that can destroy cells that control essential muscle activity such as speaking, walking, breathing, and swallowing. Normally, messages from nerve cells in the brain (also known as upper motor neurons) are transmitted to nerve cells in the brain stem and spinal cord (also known as lower motor neurons) and from them to particular muscles. When there are disruptions in these signals, the result can be gradual muscle weakening, wasting away, and uncontrollable twitching (also known as fasciculations). Eventually, the ability to control voluntary movement can be lost. MNDs may be inherited or acquired, and they occur in all age groups. In adults, symptoms often appear after age 40. In children, particularly in inherited or familial forms of the disease, symptoms can be present at birth or appear before the child learns to walk. Common MNDs include amyotrophic lateral sclerosis (ALS), progressive bulbar palsy, spinobulbar muscular atrophy, pseudobulbar palsy, primary lateral sclerosis, and progressive muscular atrophy (PMA). Other MNDs include the many inherited forms of spinal muscular atrophy, and post-polio syndrome, a condition that can affect polio survivors decades after their recovery from poliomyelitis.

As used herein, “amyotrophic lateral sclerosis” or ALS, sometimes called Lou Gehrig's disease, is a rapidly progressive, fatal neurological disease that attacks neurons responsible for controlling voluntary muscles. In ALS, both the upper motor neurons and the lower motor neurons degenerate or die, ceasing to send messages to muscles. Unable to function, the muscles gradually weaken, waste away, and twitch. Eventually the ability of the brain to start and control voluntary movement is lost. Individuals with ALS lose their strength and the ability to move their arms, legs, and body. When muscles in the diaphragm and chest wall fail, individuals lose the ability to breathe without ventilatory support.

As used herein, “patient” refers to a mammal (e.g., human) that has been diagnosed with a motor neuron disease or identified as having an increased likelihood of developing a motor neuron disease.

### BRIEF DESCRIPTION OF THE FIGURES

FIG. 1 shows the results from LCM of motor neurons from subpopulations showing differential vulnerability to degeneration in amyotrophic lateral sclerosis. (A) Approximately 40% of cervical spinal motor neurons had degenerated in the SOD1^{G93A} rats at the time of disease onset, as defined by grip strength analysis, while the number of motor neurons in the different brain stem nuclei remained unchanged. (B) Schematic figure depicting the rat brain, brainstem and spinal cord, displaying three nuclei of motor neurons along the rostrocaudal axis of the CNS which show differential vulnerability to degeneration in amyotrophic lateral sclerosis: CN3/4, CN12 and the lateral motor column of the cervical spinal cord. Motor neurons in the (C-F) CN3/4, (G-J) CN12, and (K-N) the cervical spinal cord, visualized by (C, G and K) choline acetyltransferase (ChAT) staining or (D, H and L) HistoGene staining were isolated by LCM (E, F, I, J, M and N).

FIG. 2 shows spinal motor neurons in primary culture that were protected from glutamate-induced toxicity by IGF-II. (A) The number of spinal motor neurons in primary culture was significantly decreased after the addition of 20 μM glutamate (Glu) and 100 μM of the glutamate uptake blocker PDC (P<0.001, ANOVA). Pretreatment of the cultures with IGF-II (10-100 ng/ml) for 2-4 h prior to glutamate insult protected motor neurons against the toxicity (P<0.001, ANOVA). Confocal analysis of (B and D) 150 kD neurofilament (NF) and (C and D) islet-1 expression in primary spinal cord cultures pretreated with IGF-II show the presence of large numbers of motor neurons in the cultures after 7 days of combined IGF-II and glutamate treatment. Scale bar: 50 mm (D, applies to B and C). Asterisk signifies a statistically significant difference with a P<0.01, by ANOVA. (E) The number of spinal motor neurons in primary culture was significantly decreased after the addition of 20 μM glutamate (Glu) and 100 μM of the glutamate uptake blocker PDC (P<0.001, ANOVA). However, pretreatment of the cultures with guanine deaminase prior to glutamate insult protected motor neurons against the toxicity.

FIG. 3 is a series of bar graphs showing the effect of various NSAIDs on IGF-II mRNA expression in primary spinal cord cultures.

FIG. 4 is a series of bar graphs showing the effect of various NSAIDs on IGF-II mRNA expression in primary spinal cord cultures.

FIG. 5 is a series of bar graphs showing the effect of various antihypertensive agents on IGF-II mRNA expression in primary spinal cord cultures.

FIG. 6 is a series of bar graphs showing the effect of various antihypertensive agents on IGF-II mRNA expression in primary spinal cord cultures.

FIG. 7 is a series of schematic diagrams showing vector construct used in the high throughput luciferase reporter assays in which the luciferase reporter gene is placed under the operational control of the IGF-II P4 promoter. The IGF-II P4 promoter sequence is provided (SEQ ID NO: 13).

FIG. 8 is a bar graph showing the number of motor neurons in a cell population after treatment with each indicated compound, as a percentage of the number of untreated control population, demonstrating the effect of IGF-II up-regulating drugs on low level progressive glutamate excitotoxicity in primary spinal cord cultures.

FIG. 9 is a bar graph showing the comparable luminescence as a result of the high throughput luciferase reporter assays between hydrocortisone, a known P4 activator, and Vardenafil HCl, a candidate drug discovered in the small library screening.

FIG. 10 is a schematic diagram showing the combination P3P4 vector construct to be used in a high throughput luciferase reporter assay in which the luciferase reporter gene is placed under the operational control of the IGF-II promoters P3 and P4. The P3P4 promoter sequence is provided (SEQ ID NO:14).

FIG. 11 is a representation of the structure of Vardenafil HCl.

### DETAILED DESCRIPTION

The present invention relates generally to methods for treating motor neuron diseases. Motor neuron diseases may be treated by administering to a patient in need thereof, any one or more of the therapeutic agents (or therapeutic agents from the classes of therapeutic agents) disclosed herein. Preferably, the therapeutic agents increase the expression of IGF-II and/or guanine deaminase in the motor neurons of the patient.

### Formulation and Administration of Therapeutic Agents

Therapeutic agents of the invention can be administered to a patient, e.g., a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York.

Pharmaceutical formulations of a therapeutically effective amount of a peptide agent or candidate compound of the invention, or pharmaceutically acceptable salt-thereof, can be administered orally, parenterally (e.g. intramuscular, intraperitoneal, intravenous, or subcutaneous injection), or by intrathecal or intracerebroventricular injection in an admixture with a pharmaceutically acceptable carrier adapted for the route of administration.

Methods well known in the art for making formulations are found, for example, in Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the proteins of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

The amount of active ingredient in the compositions of the invention can be varied. One skilled in the art will appreciate that the exact individual dosages may be adjusted somewhat depending upon a variety of factors, including the protein being administered, the time of administration, the route of administration, the nature of the formulation, the rate of excretion, the nature of the subject's conditions, and the age, weight, health, and gender of the patient. Generally, dosage levels of between 0.1 mg/kg to 100 mg/kg of body weight are administered daily as a single dose or divided into multiple doses. Desirably, the general dosage range is between 250 mg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

If more than one agent is employed, each agent may be formulated in a variety of ways that are known in the art. Desirably, the agents are formulated together for the simultaneous or near simultaneous administration of the agents. Such co-formulated compositions can include the two agents formulated together in the same pill, capsule, liquid, etc. It is to be understood that, when referring to the formulation of such combinations, the formulation technology employed is also useful for the formulation of the individual agents of the combination, as well as other combinations of the invention. The individually or separately formulated agents can be packaged together or separately, or may be co-formulated.

Generally, when administered to a patient, the timing dosage of any of the therapeutic agent(s) will depend on the nature of the agent, and can readily be determined by one skilled in the art. Each agent may be administered once or repeatedly over a period of time (e.g., including for the entire lifetime of the patient).

### EXAMPLES

The present methods and kits, thus generally described, will be understood more readily by reference to the following examples, which are provided by way of illustration and are not intended to be limiting of the present methods and kits.

### Example 1

### Increased IGF-II and/or Guanine Deaminase Expression Protects Motor Neurons from Degeneration

Laser capture microscopy was used to isolate motor neurons from CN3/4, CN12 and the LMC of the cervical spinal cord of the normal rat and performed an analysis of the entire rat transcriptome. Differential expression of selected genes with implications for motor neuron vulnerability was confirmed by localization of the resulting proteins. Functional in vitro analysis revealed that the CN3/4-specific genes IGF-II and Guanine Deaminase protect motor neurons from glutamate-induced toxicity.

### Materials and Methods

Animal Procedures.

All animal procedures were performed in accordance with the National Institute of Health guidelines and were approved by the Animal Care and Use Committee at McLean Hospital, Harvard Medical School. Animals were housed according to standard conditions, with access to food and water ad libitum and a dark/light cycle of 12 h.

Analysis of Disease Onset in the SOD1^{G93A} Rat Model of ALS.

Transgenic rats over-expressing mutant SOD1 (SOD1^{G93A}) were used as a model of ALS. Disease onset in these animals was evaluated by grip strength analysis (Grip strength meter, Columbus Instruments), with weekly fore limb and hind limb strength measurements, bi-weekly weight measurements and visual observation of ambulatory behavior. Onset of disease was determined as the time point when animals had lost 81±5.5% of their peak grip strength in the most severely affected limbs, which then showed a slight dragging.

Tissue Preparation.

Presymptomatic, 60-day-old female SOD1^{G93A} transgenic and wild-type (wt) litter-mates (Taconic) and symptomatic SOD1^{G93A} rats and age-matched wt litter-mates were anesthetized with sodium pentobarbital (150 mg/kg i.p.). For LCM and real-time PCR, tissues were removed, snap-frozen in 2-methylbutane (−60° C.), sectioned (12 μM coronal sections), mounted onto LCM slides (Arcturus Engineering, Inc, Mountain View, Calif.) and stored at −70° C. For immunohistochemistry, animals were perfused intracardially with 100 ml heparinized saline and 200 ml 4% paraformaldehyde. Brains, brain stems, and spinal cords were dissected, postfixed for 6 h, cryoprotected in 20% sucrose, sectioned (30-40 μM) and stored at −70° C.

Quantification of Differential Motor Neuron Loss in the SOD1^{G93A} Rat.

The number of motor neurons present in the CN3/4, trigeminal nucleus (CN5), facial nucleus (CN7), CN12 and in the lateral motor column across C2 and C3 segments in the cervical spinal cord from 60-day-old SOD 1^{G93A} rats, 60-day-old wild-type litter mates and symptomatic SOD1^{G93A} rats and age-matched wild-type litter mates were quantified. Sections were incubated with blocking buffer (phosphate buffered saline, 10% normal donkey serum or normal goat serum and 0.1% Triton-X100) for 1 h. Sections were incubated overnight at 4° C. with primary antibody against choline acetyltransferase (1:750, Millipore). Sections were washed in phosphate buffered saline and incubated with a biotinylated secondary antibody (1:300; Vector Laboratories, Burlingame, Calif.) for 1 h at room temperature, followed by incubation in streptavidin-biotin complex (Vectastain ABC kit Elite, Vector laboratories) for 1 h and visualized by incubation in 3,3′-diaminobenzidine solution (Vector Laboratories). The number of cranial and cervical spinal cord choline acetyltransferase positive motor neurons was quantified.

Quick Immunostaining and Dehydration of Sections for Laser Capture Microdissection.

To visualize motor neurons for LCM, sections on slides were fixed in 75% ethanol for 1 min, washed in distilled water for 2 min, stained for 4 min in HistoGene staining (Arcturus) and washed again for 30 s in distilled water. The sections were dehydrated for 30 s in 75% ethanol, 2 min in 95% ethanol, 1 min in 100% ethanol and 5 min in xylene, air dried and placed into the Veritas LCM (Arcturus).

Laser Capture Microdissection of Motor Neurons.

An Arcturus Veritas LCM System was used to isolate motor neurons from the CN3/4, CN12 and the LMC of the csc (C2 and C3 segments) of normal rats onto CapSure Macro LCM caps (Arcturus). 500-1,000 motor neurons were isolated from each subpopulation and each individual animal (n=4-5). Settings were optimized to capture nucleus and cytosol from the motor neurons, but minimizing inclusion of surrounding tissues.

RNA Preparation, Amplification and Oligo-Microarray Analysis.

RNA was purified from 250-500 motor neurons isolated from CN3/4, CN12 or csc (PicoPure RNA isolation kit, Arcturus), and amplified (aRNA) (RiboAmp RNA amplification kit, Arcturus). aRNA quality was analyzed (Agilent 2100 Bioanalyzer, Agilent technologies) and hybridized to whole rat genome oligo microarrays (Rat Genome 230 2.0 Array, Affymetrix). The microarray study consisted of a comparison between motor neurons isolated from CN3/4, CN12 or csc. Each group constituted of 4-5 replicates (arrays). The data set was initially analyzed using Gene Pattern (available on-line at the Massachusetts Institute of Technology), with ×500 scaling normalization and removal of absent calls. Genes with differential expression among the different subpopulations were identified by in pair comparison (Microsoft Excel, two-tailed distribution and two sample equal variance, homoscedastic, p<0.05). Genes were sorted by fold-change. For analysis of gene variance within and between motor neuron subpopulations, genes with a standard deviation >1 were removed, resulting in an analysis of 19,722 genes (CN3/4), 21,560 genes (CN12) and 16,843 genes (csc) and 12,773 genes (all three nuclei) for the cross-comparison of all three subpopulations. The Multiexperiment viewer (MeV) of TM4 was used for correspondence analysis (COA) using Euclidean distance and average linkage clustering. For COA the K-nearest neighbor algorithm was utilized and the number of neighbors for KNN imputation was set to 10. For the hierarchical clustering and Gene distance matrix (GDM) analysis based on Euclidian distance, genes with significant differential expression (1,968 genes) were extracted by ANOVA, p≦0.01 (based on F-distribution). The Affymetrix microarray raw data was further processed in Gene Pattern by thresholding/ceiling and variation filter and differentially expressed genes were identified by a comparative marker selection based on a 2-sided t-test using 1,000 permutations. Genes differentially expressed between CN3/4, CN12 and csc, p≦0.05, were selected and visualized in Heat maps, gene lists and annotations. All gene lists were annotated using the DAVID Bioinformatics Database Gene Id Conversion Tool and NCBI Entrez gene database and BLAST tool. The functional annotation chart tool in DAVID was utilized to detect differences in gene groups between motor neuron subpopulations. Classification stringency in this analysis was set using the medium parameter and the similarity threshold to 0.5.

Preparation of primary spinal cord Cultures and In Vitro Analysis of Neuroprotection.

Time-pregnant Sprague Dawley wild-type rats were anesthetized, decapitated and E15.5 embryos collected, decapitated and spinal cords isolated in Hanks balanced salt solution (HBSS, Invitrogen). Dissections of spinal cords were done carefully to avoid the inclusion of somites or other external tissues. Cells were dissociated by gentle trituration and incubation with papain (Worthington Biochemical Corporation). Cells were cultured for 6 days in Neurobasal media (Invitrogen) containing 10% fetal bovine serum (FBS, Fisher scientific), 1×B27 supplement (Invitrogen), 500 μM glutamine (Invitrogen), 25 mM mercaptoethanol (Invitrogen), Penicillin-Streptomycin (Invitrogen) or in DMEM/F12 (Invitrogen) containing 5% FBS, 1×N2 supplement A (Stem Cell Technologies), glucose (0.36%, Sigma), bovine serum albumin (0.25%, Invitrogen), Penicillin-Streptomycin (Invitrogen). Either culture media could maintain spinal cord cultures containing motor neurons. This 6-day culture period was developed to allow astrocytes time to proliferate in vitro and motor neurons to form an interconnected network prior to exposure to glutamate and the glutamate uptake blocker, L-trans-2,4-Pyrrolidine-2,4-dicarboxylic acid (PDC). After the 6 days of culture, glutamate toxicity was induced by the addition of 20 μM glutamate and 100 μM PDC for 4-7 days. For analysis of neuroprotection, the glutamate challenge was preceded by a 2-4 h pretreatment with 1-100 ng/ml recombinant IGF-II (R&D Systems) or 100 ng/ml Guanine Deaminase (MP Biomedicals, LLC, Solon, Ohio). Cultures were subsequently maintained for an additional 4-7 days. Identification of motor neurons was done by staining fixed cultures using antibodies against islet-1/2 (1:500 when antibodies were used in combination and 1:100 when used separately, 39.4D5, 40.2D6, Developmental Studies Hybridoma Bank (DSHB), University of Iowa), Neurofilament (NF) (150 kD) (1:500) and ChAT (1:500, Millipore) and MNR2/HB9 (1:100, 81.5C10, DSBH).

Immunofluorescent Staining and Stereological Procedures.

For immunofluorescent staining, coverslips/sections were rinsed with phosphate buffered saline and incubated with blocking buffer (see above) for 1 h. Coverslips/sections were then incubated overnight at 4° C. with primary antibodies in blocking buffer. The following antibodies were used: mouse anti-islet-1/2 and rabbit anti-neurofilament (see above), rabbit anti-peripherin (1:100), mouse anti-tyrosine hydroxylase (1:1000), mouse anti-NeuN (1:1000, Millipore), rabbit anti-G protein-coupled inwardly rectifying potassium channel 2 (1:80, Alomone Laboratories), rabbit anti-guanylate cyclase soluble subunit alpha-3 (Gucy1a3) (1:60, Abgent), rabbit anti-placental growth factor (1:30, Proteintech group), rabbit anti-IGF-II (1:100, R&D systems), rabbit anti-early growth response protein 1 (1:100), goat anti-cypin (Guanine Deaminase) (A-20, 1:100, Santa Cruz Biotechnology) and mouse anti-glial fibrillary acidic protein (1:1000, Sigma). Localization of the proteins was done on multiple sections along the cervical spinal cord in multiple animals. The coverslips/sections were then incubated with Alexafluor secondary antibodies for 1 h and rinsed. Hoechst 33342 (4 mg/ml) was used for counterstaining Confocal analysis was performed using a Zeiss LSM510/Meta Station (Thornwood, N.Y.), with optical thickness kept to a minimum and orthogonal reconstructions obtained. The effect of glutamate, IGF-II and/or guanine deaminase on motor neuron survival was carefully evaluated, as was the co-localization of motor neuron markers.

Quantitative PCR.

Quantitative PCR utilizing SYBR green I (Molecular Probes, Eugene, Oreg.) or specific Taqman probes was used to confirm differential gene expression. RNA extracted from 100-200 LCMed motor neurons were reverse transcribed using Superscript III (Invitrogen). 2 μl of diluted cDNA was amplified (DNA Engine Opticon real-time PCR machine, Bio-Rad), in 25 ml reactions containing 900 nM primer and 250 nM probe in Taqman Universal Master Mix (Applied Biosystems). Reactions were run in duplicate and each gene analyzed in at least three different animals. Reaction parameters were as follows: 50° C. for 2 min, 95° C. for 10 min, and 40 cycles of 95° C. for 15 s and 60° C. for 1 min. Controls lacking template and/or Superscript were included. The absolute copy number for each gene was quantified using a standard curve from human GAPDH plasmid and amplified products were normalized against rat GAPDH expression. Primers and probes (Integrated DNA technologies) were as follows: rat GAPDH: Forward primer: TCCGTTGTGGATCTGA (SEQ ID NO: 1); Reverse primer: CACCACCTTCTTGATGTC (SEQ ID NO: 2); Probe: 6′FAM-ATGCCGCCTGGAGAAACC TGCC-BHQ-1 (SEQ ID NO: 3); Peripherin: Forward primer: CACAACCTGGTGCTCTT (SEQ ID NO: 4); Reverse primer: CTTCGTGTAGCTTCTTGA (SEQ ID NO: 5); Probe: 6′FAM-CTTCGTGTAGCTTCTTGA-BHQ-1 (SEQ ID NO: 6); IGF-II: Forward primer: GACACGCTTCAGTTTG (SEQ ID NO: 7); Reverse primer: AAGCAGCACTCTTCCA (SEQ ID NO: 8); Probe: 6′FAM-CTGTTCGGACCGCGGCTTCTA-BHQ-1 (SEQ ID NO: 9), Human GAPDH: Forward primer: AAGCAGCACTCTTCCA (SEQ ID NO: 10); Reverse primer: GGGTGCTAAGCAGTTG (SEQ ID NO: 11); Probe: 6′ FAM-TGACAACAGCCTCAAGATC ATCAGCA-BHQ-1 (SEQ ID NO: 12).

### Results

Quantification of Motor Neuron Survival In Vitro.

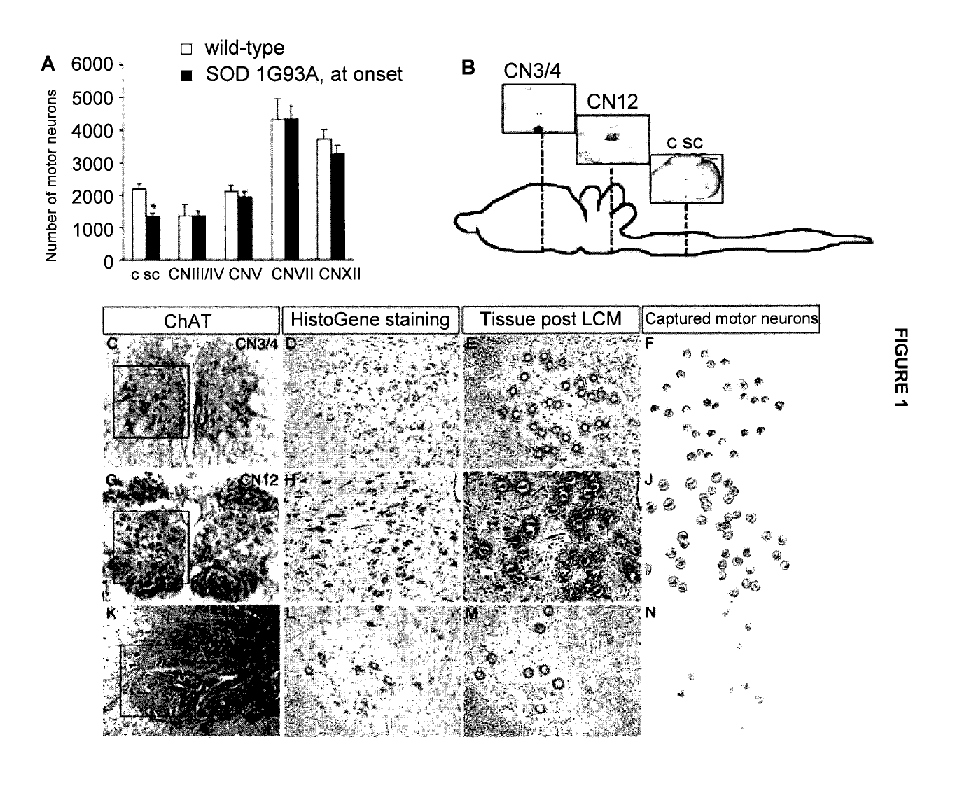

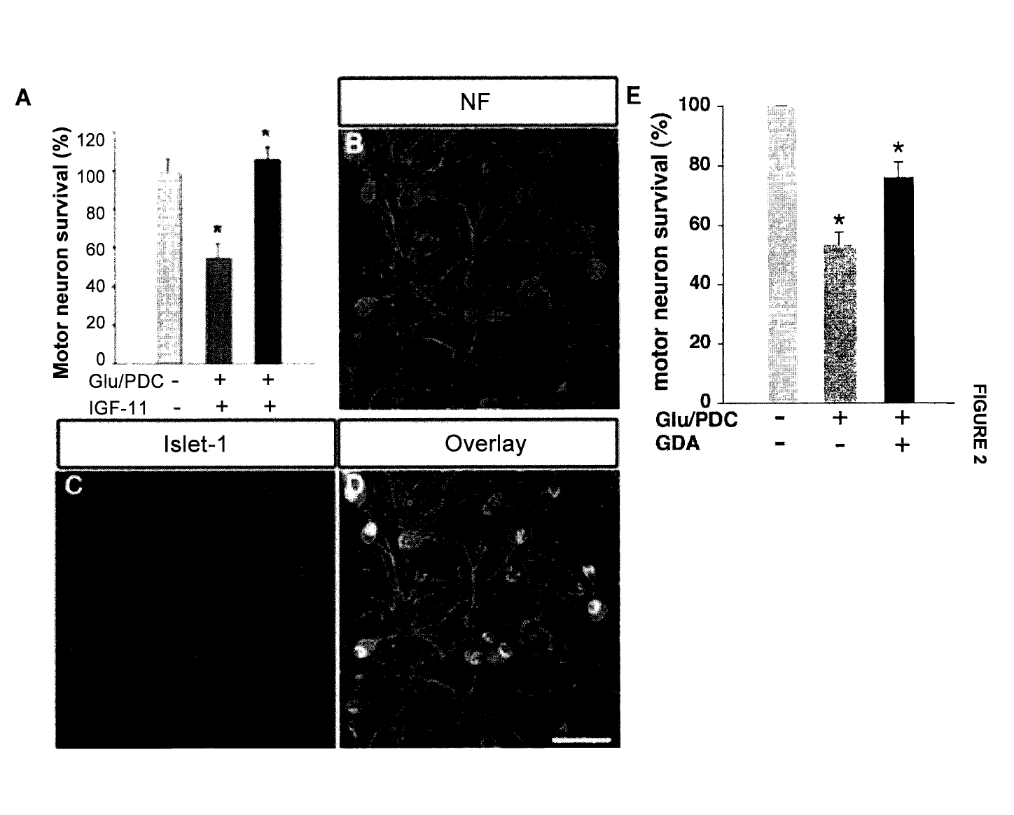

Quantification of choline acetyl transferase (ChAT) stained sections showed that there was no difference in the number of motor neurons in CN3/4, CN5 or CN7 between symptomatic SOD1G^{93A} rats and wild-type littermates (FIG. 1A, B). However, the number of motor neurons in CN12 appeared to be declining in the symptomatic SOD1G^{93A} rats compared to the wild-type littermates, with 3,285±250 versus 3,730±287 motor neurons, respectively (p=0.0513, t-test) (FIG. 1A). Furthermore, there were vacuolar inclusions present in both CN5 and CN12, indicating that these nuclei were affected by disease (data not shown). Based on our analysis and previous analyses showing that CN12 shows a significant loss of motor neurons in mutant SOD1 mouse models (Haenggeli and Kato, 2002; Nimchinsky et al., 2000), we deemed the hypoglossal motor neurons to be slightly affected. Motor neurons of CN3/4 remained unaffected in the symptomatic SOD1G^{93A} rat, whereas approximately 40% of the motor neurons in the cervical spinal cord were lost at the time of motor symptom onset, with 1,342±102 motor neurons present in symptomatic rats versus 2,204±134 motor neurons in wild-type littermates (p=0.0033, t-test) (FIG. 1A-B).

Microarray Analysis of Motor Neurons Showed High Reproducibility and Specificity to the Anatomical Nuclei.

Based on the differential motor neurons loss in motor neuron diseases, we isolated individual motor neurons from CN3/4 (do not degenerate in amyotrophic lateral sclerosis, spinobulbar muscular atrophy or spinal muscular atrophy), CN12 (show vulnerability in amyotrophic lateral sclerosis and spinobulbar muscular atrophy) and from the lateral motor column of the cervical enlargement of the spinal cord (degenerate in all three diseases) using LCM in wild-type rats (FIG. 1A-N). The RNA isolated from motor neurons was hybridized to whole genome rat arrays. The gene expression data showed that individual replicates within a motor neuron nucleus were highly reproducible with an average Pearson's correlation of 0.93 for CN3/4, 0.94 for CN12 and 0.93 for the cervical spinal cord. When motor neurons from different groups were compared, the average Pearson's correlation was 0.91 for CN12 versus cervical spinal cord, 0.89 for CN3/4 versus CN12 and 0.86 for CN3/4 versus cervical spinal cord cross comparisons. Additionally, correspondence analysis, hierarchial clustering and gene distance matrix confirmed that individual samples within each nucleus clustered together and showed that motor neurons located in CN12 and in the lateral motor column of the cervical spinal cord had the most similar transcriptomes, whereas motor neurons of CN3/4 showed a more different gene regulation pattern. Within the heat map, the locations of peripherin, which was predominantly expressed in motor neurons of the cervical spinal cord, and IGF-II and guanine deaminase, which were restricted to CN3/4 motor neurons, have been indicated.

Genes of the Hox cluster provide positional information needed for spatial and temporal pattering of the vertebrate body axis. The known differential expression of the Hox genes, along the anterior posterior axis of the developing hindbrain and spinal cord, was used to validate the microarray data further. Hox A genes in positions 1 and 2, with anterior limits within the midbrain/hindbrain were expressed in all three motor neuron nuclei, as would be expected. Certain Hox genes were identified only in CN12 and the cervical spinal cord as expected based on their known expression in brain stem and spinal cord, but lack thereof in midbrain. Other Hox genes, with anterior limits at cervical spinal cord levels, were consequently only identified in motor neurons of the cervical spinal cord. There were large differences in the number of regulated genes involved in endoplasmatic reticulum function, mitochondria, ubiquitination, apoptosis regulation, nitrogen metabolism, calcium regulation, transport, cell adhesion and growth. There were also large differences in the number of genes involved in transcription, RNA metabolic and biosynthetic processing, RNA binding, RNA splicing and regulation of translation, functions which have recently been implicated in amyotrophic lateral sclerosis. Furthermore, we identified IGF-II to be preferentially expressed in motor neurons of the CN3/4. IGF-II has been suggested to protect spinal motor neurons from degeneration in a mouse model of amyotrophic lateral sclerosis.

The present finding that IGF-II is preferentially expressed within CN3/4 motor neurons explains the resistance of these cells to degeneration. Functional annotation and pathway analysis showed that several genes, including catalase, neurofilaments, protein phosphatase 3 and tumor protein p53, shown to be involved in amyotrophic lateral sclerosis pathogenesis, were more highly expressed in motor neurons of the cervical spinal cord. Furthermore, investigation of ubiquitin mediated proteolysis, a process thought to be involved in the pathogenesis of motor neuron diseases showed that multiple genes were expressed at higher levels in spinal cord motor neurons.

Confirmation of Differential Expression in Specific Anatomical Motor Neuron Nuclei.

Immunofluorescence of the resulting proteins of genes identified as differentially expressed among motor neuron subpopulations displaying differential vulnerability to degeneration confirmed their specific localization and differential expression. The intermediate neurofilament peripherin protein showed a preferential expression within spinal motor neurons, consistent with the mRNA expression. Placental growth factor protein was predominantly localized to spinal motor neurons, consistent with their microarray data. IGF-II mRNA and protein were restricted to motor neurons of CN3/4.

Guanine deaminase mRNA and protein were restricted to CN3/4 motor neurons. Guanine deaminase was also expressed within the striatum. The soluble protein Gucy1a3's mRNA and protein were mainly expressed in motor neurons of CN3/4, but were also detectable in spinal motor neurons. Gucy1a3 protein was also expressed in non-motor neurons within and surrounding CN3/4. Early growth response 1 protein was mainly expressed in CN3/4 motor neurons, consistent with the mRNA expression. The G protein-coupled inwardly rectifying potassium channel 2 mRNA and protein were predominantly expressed in CN3/4 motor neurons. In the cervical spinal cord, the expression appeared more variable, with some neurons displaying a high and others a somewhat lower level of the protein.

The CN3/4-Restricted Genes IGF-II and Guanine Deaminase Protected Spinal Motor Neurons from Glutamate-Induced Toxicity.

For analysis of possible neuroprotective properties of differentially expressed candidate genes on somatic motor neurons we developed a primary embryonic spinal cord culture system. The cultures initially contained a majority of neurons, but also a smaller population of astrocytes, which continuously proliferated and thereby constituted the majority of cells at the later parts of the culture time. The presence of cell types other than motor neurons provided trophic support, enabling culturing without the addition of growth factors that are necessary if motor neurons are to be cultured alone. Motor neurons were present at all times in the culture and displayed large neuritic networks as the culture time progressed. The motor neurons had a healthy appearance and expressed neurofilament and islet-1. Islet-1-positive cells also expressed homeobox 9 (98.4±1.5% of islet-1 positive cells were homeobox 9 positive) and choline acetyltransferase, confirming their motor neuron identity. At Day 13 of the culture, 9.7±5.3% of all the cells in the culture were motor neurons (homeobox 9 positive, islet-1 positive).

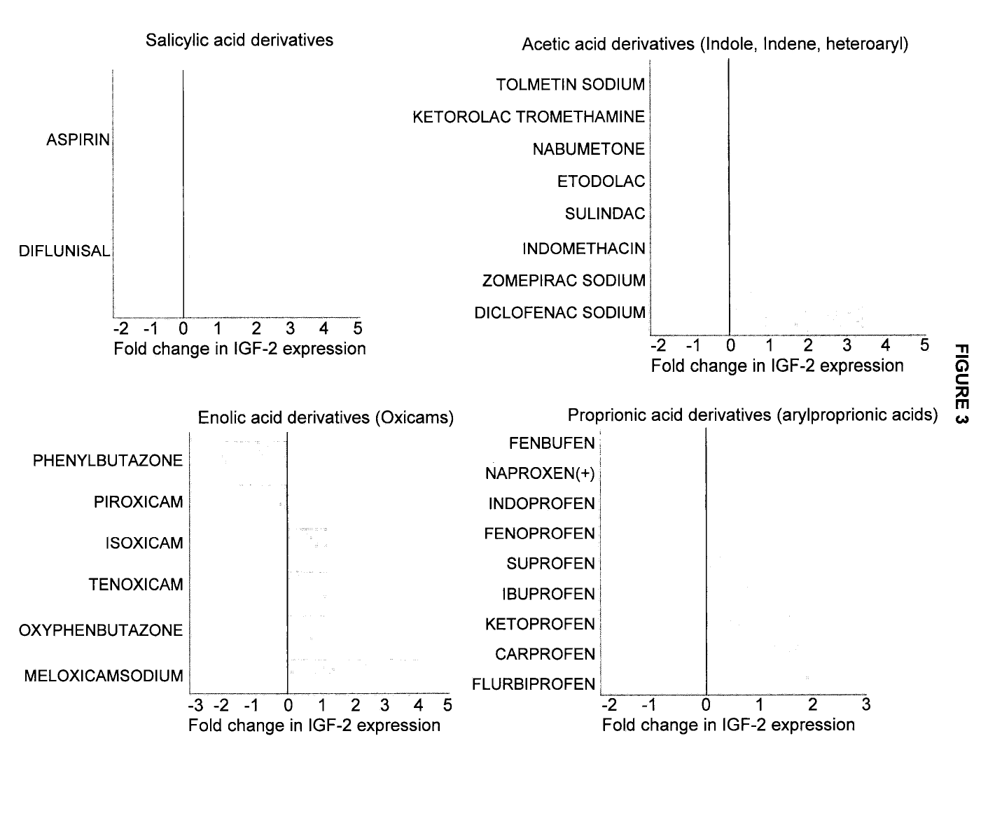

Glutamate toxicity could be a general downstream event of degeneration in motor neuron disease. Addition of glutamate (20 μM) and a general glutamate uptake blocker (PDC, 100 μM) induced motor neuron toxicity (FIG. 2A). We selected the CN3/4-restricted genes IGF-II and guanine deaminase for analysis of neuroprotective properties, based on their high differential expression and predominant expression in protected motor neurons and specific cellular functions. We hypothesized that the endogenous expression of IGF-II and/or guanine deaminase within CN3/4 motor neurons might protect these cells from glutamate toxicity.

IGF-II is a survival factor for motor neurons in some instances and guanine deaminase is a protein important for dendritic branching and synaptic function, but it was not known if either of these proteins could help motor neurons resist high levels of glutamate. Because IGF-II and guanine deaminase are both present extracellularly, which may be of significance and benefit for therapeutic development, we added either of these proteins exogenously to primary spinal cord cultures prior to glutamate insult. Pretreatment with IGF-II at 10-100 ng/ml concentrations protected motor neurons from glutamate-induced toxicity (FIG. 2A-D). Confocal analysis of IGF-II pretreated cultures exposed to glutamate for 7 days show healthy motor neurons expressing neurofilament and islet-1 (FIG. 2B-D). Preincubation of the spinal cultures with guanine deaminase at 100 ng/ml concentrations also protected motor neurons from glutamate-induced toxicity (FIG. 2E).

### Discussion

Relative Vulnerability and Comparative Analysis of Differential Gene Expression Among Motor Neuron Subpopulations.

Factors intrinsic to motor neurons appear crucial for initiation of motor neuron degeneration in amyotrophic lateral sclerosis and perhaps also in spinobulbar muscular atrophy and spinal muscular atrophy. To understand why disease is initiated in some, but not all motor neurons we analyzed the gene expression profiles of motor neurons isolated from CN3/4 (unaffected in amyotrophic lateral sclerosis, spinobulbar muscular atrophy and spinal muscular atrophy), CN12 (affected in amyotrophic lateral sclerosis and spinobulbar muscular atrophy) and the lateral motor column of the cervical spinal cord (highly affected in amyotrophic lateral sclerosis, spinobulbar muscular atrophy and spinal muscular atrophy). We studied motor neurons using LCM, which allows for isolation of neurons from adult animals and does not require any genetic manipulation.

The foregoing analyses demonstrated that motor neurons in CN12 and the lateral motor column of the cervical spinal cord had more commonality in gene expression levels than those in CN3/4. This higher degree of clustering of CN12 and cervical spinal cord motor neurons could be related to parameters such as the size of the neurons, the lengths of their projections and their respective muscle targets. These analyses also revealed that all three motor neuron subpopulations displayed distinct profiles and exhibited genes with unique expression. The identification of differences in Hox gene expression levels in motor neurons isolated from the three anatomical nuclei along the anterior-posterior (A-P) axis of the adult rat matched that of the developing embryo and validated the microarray data. This differential Hox gene expression pattern in the adult nervous system indicates that these genes might be important for maintenance of phenotype in addition to providing positional information during development. Consistent with such a role, adult expression of the Hox-like homeoprotein pancreatic and duodenal homeobox 1 (Pdx1) is necessary for the maintenance of pancreatic cells and prospero homeobox protein 1 for lymphatic endothelial cells. Comparison of groups of genes revealed differences in regulation of genes involved in endoplasmatic reticulum and mitochondrial functions, ubiquitination, apoptosis regulation, nitrogen metabolism, calcium regulation, transport and growth.

Differential Expression of Genes Related to Motor Neuron Vulnerability.

We identified peripherin to be predominantly expressed in spinal motor neurons. Over-expression of peripherin, results in defective axonal transport of neurofilament proteins and late-onset motor neuron degeneration. Elevated levels of peripherin splice forms have been detected in spinal cords of patients with familial and sporadic amyotrophic lateral sclerosis. Mutations in the peripherin gene are associated with a small percentage of amyotrophic lateral sclerosis cases. Consequently, a higher level of peripherin within specific motor neurons might predispose these cells to degenerative events.

We also identified several genes as selectively expressed within motor neurons of CN3/4, which could play protective roles, e.g. Gucy1a3, early growth response protein 1, IGF-II, and guanine deaminase. Gucy1a3 functions as the main receptor for nitric oxide. Motor neurons from mutant SOD1 mice show increased susceptibility to exogenous nitric oxide, through upregulation of Fas ligand and subsequent Fas receptor activation. The activation of Fas receptor leads to further nitric oxide synthesis and it has been proposed that chronic low-level activation of the Fas/nitric oxide feedback loop may underlie the progressive motor neuron loss that characterizes familial amyotrophic lateral sclerosis. The presence of Gucy1a3 within motor neurons of CN3/4 suggests that these cells will contain less unbound nitric oxide and, as a consequence, might show a lower level of Fas activation. Furthermore, the CN3/4-restricted gene early growth response protein 1 can confer resistance to apoptotic signals by inhibiting Fas expression, and thereby leading to insensitivity to Fas ligand. The higher expression of early growth response protein 1 within motor neurons of CN3/4 could help to explain further why these cells are not affected by degeneration in amyotrophic lateral sclerosis.

In addition, the restricted expression of IGF-11 to CN3/4 motor neurons could prove beneficial to these cells. IGF-II can act as a survival factor for motor neurons and can support regeneration of motor axons after nerve injury and during normal development. Guanine deaminase catalyses the conversion of guanine to xanthine. Analysis in hippocampal neurons has shown that guanine deaminase can regulate post-synaptic sorting and promote dendritic branching. The gene TDP-43 can promote dendritic branching, but amyotrophic lateral sclerosis-associated mutations in TDP-43 attenuates the dendritic function. Therefore, a high expression of guanine deaminase, a protein important for dendritic branching and synaptic function, is protective to motor neurons.

Functional Analysis Revealed Neuroprotective Properties of the CN3/4-Restricted Genes IGF-II and Guanine Deaminase.

Motor neuron toxicity and protection in response to glutamate was assayed in a system containing neurons and astrocytes. Glutamate toxicity was utilized since it is considered a downstream event in motor neuron degeneration. Motor neurons are usually protected from high levels of glutamate in vivo by surrounding astrocytes. However, astrocytes in the spinal cords of patients with amyotrophic lateral sclerosis and lower motor neuron disease and in mutant SOD1 mice and rats have been shown to lose the expression of the focal glutamate transporter excitatory amino acid transporter 2, which could decrease their ability to sequester glutamate. In the foregoing assays, excitatory amino acid transporters were blocked to mimic glutamate over-load in motor neuron disease and to create a reliable tool in predicting substances that can protect motor neurons in vivo. Subsequently, the effects of the CN3/4-specific genes IGF-II and guanine deaminase were tested in the assays and it was found that IGF-II blocked glutamate-induced motor neuron loss completely, while guanine deaminase considerably decreased the loss of motor neurons.

These data demonstrate that motor neuron diseases may be treated with compounds that either increase the endogenous expression of IGF-II or mimic the effect of IGF-II on a cell. Alternatively, or in addition to the IGF-II effect, motor neuron disease may be treated with compounds that either increase the endogenous expression of guanine deaminase or mimic the effect of guanine deaminase on a cell.

### Example 2

### Identification of Therapeutic Compounds that Increase IGF-II Expression for the Treatment of Motor Neuron Disease

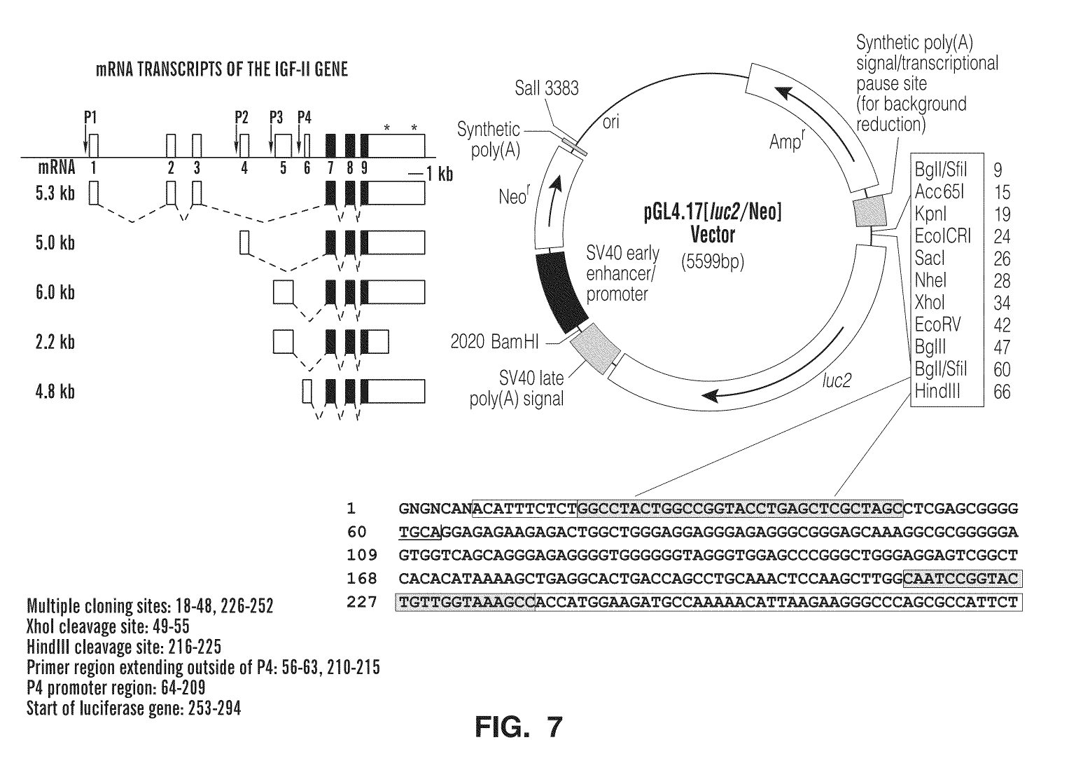

Two libraries of FDA-approved drugs (NINDS and Harvard LDDN) were screened for their ability to up-regulate IGF-II expression. Candidate compounds were screened in an IGF-II expression assay using a qPCR detection methodology in primary spinal cord cultures or using a luciferase reporter assay. In the initial screening, therapeutic candidates were identified as those that increased IGF-II expression by at least 2-fold. FIG. 7 shows the luciferase report construct, under the control of the IGF-II promoter, that was used in the assay.

The P4 promoter is transfected into the pGL4.17 [luc/Neo] vector. The insertion sequence is

| (SEQ ID NO. 13) | |
|---|---|
| 1 | GNGNCANACA TTTCTCTGGC CTACTGGCCG GTACCTGAGC TCGCTAGCCT CGAGCGGGGT |
| 61 | GCAGGAGAGA AGAGACTGGC TGGGAGGAGG GAGAGGGCGG GAGCAAAGGC GCGGGGGAGT |
| 121 | GGTCAGCAGG GAGAGGGGTG GGGGGTAGGG TGGAGCCCGG GCTGGGAGGA GTCGGCTCAC |
| 181 | ACATAAAAGC TGAGGCACTG ACCAGCCTGC AAACTCCAAG CTTGGCAATC CGGTACTGTT |
| 241 | GGTAAAGCCA CCATGGAAGA TGCCAAAAAC ATTAAGAAGG GCCCAGCGCC ATTCT. |

The vector comprises multiple cloning sites at by 18-48, an Xhol cleavage site at by 49-55, a HindIII cleavage site at by 216-225, a primer region extending outside of P4 at by 56-63 and 210-215, the P4 promoter region at by 64-209, and the start of the luciferase gene at by 253-295.

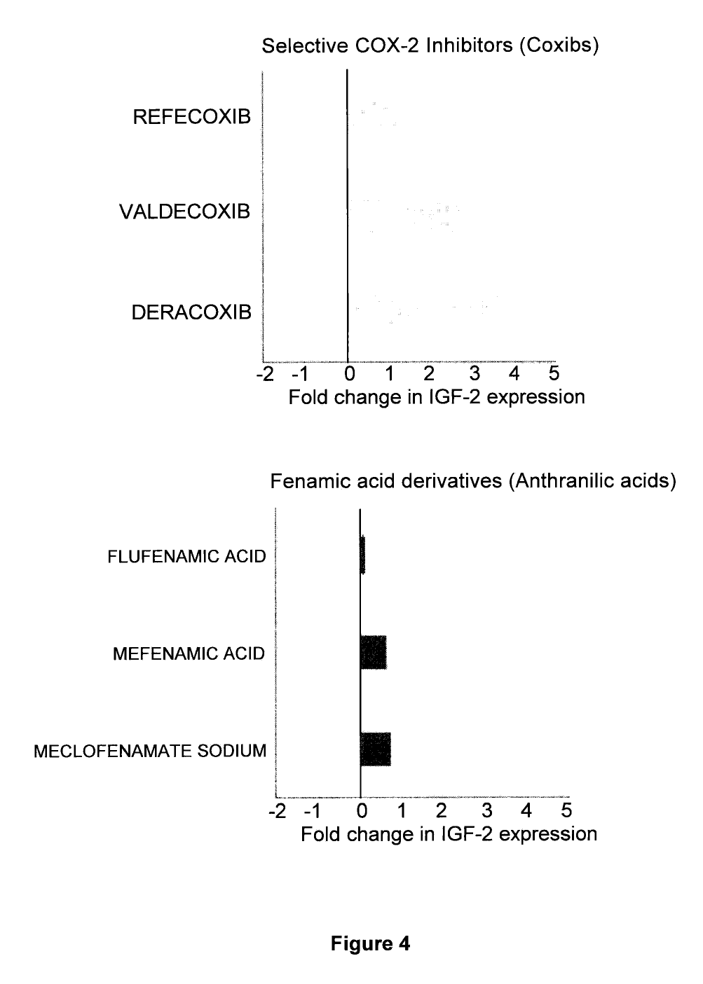

FIGS. 3 and 4 show the results for the induction of IGF-II expression in a series of NSAIDs including salicylic acid derivatives, acetic acid derivatives (indole, indene, heteroaryl derivatives), enolic acid derivatives (oxicams), proprionic acid derivatives (arylproprionic acids), selective COX-2 inhibitors (Coxibs) and fenamic acid derivatives (anthranilic acids). Notably, diclofenac (3.4±1.3 fold induction) and flurbiprofen (2.5±0.5 fold induction) showed good activity. Each of these therapeutic agents is known to be CNS active. Other therapeutic agents showing strong IGF-II inducing activity include diflunisal, meloxicam, carprofen, valdecoxib, and deracoxib.

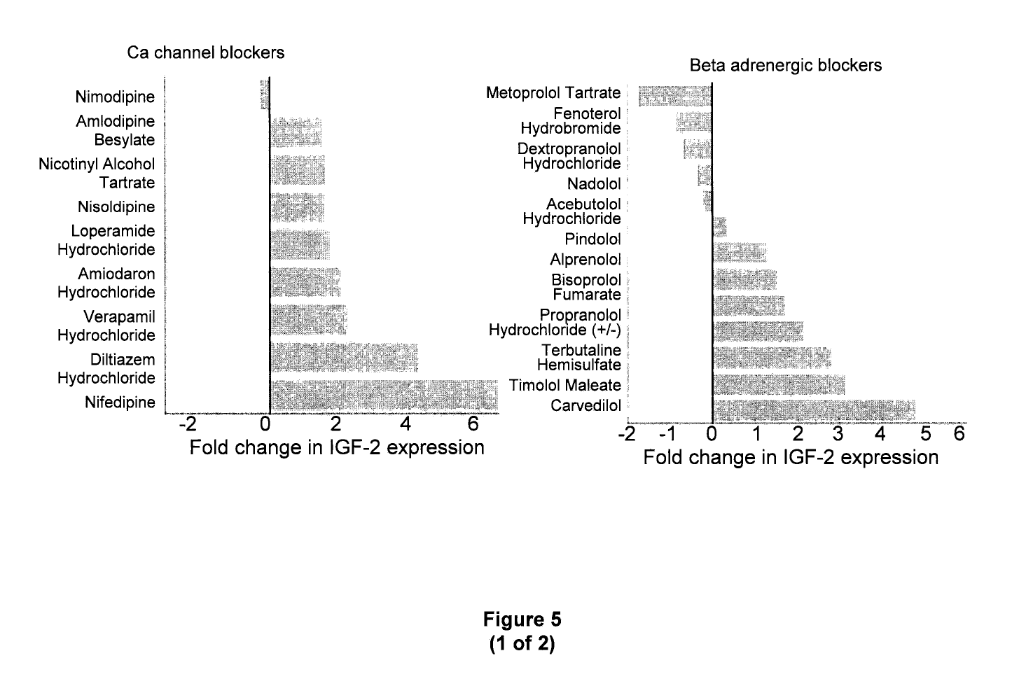

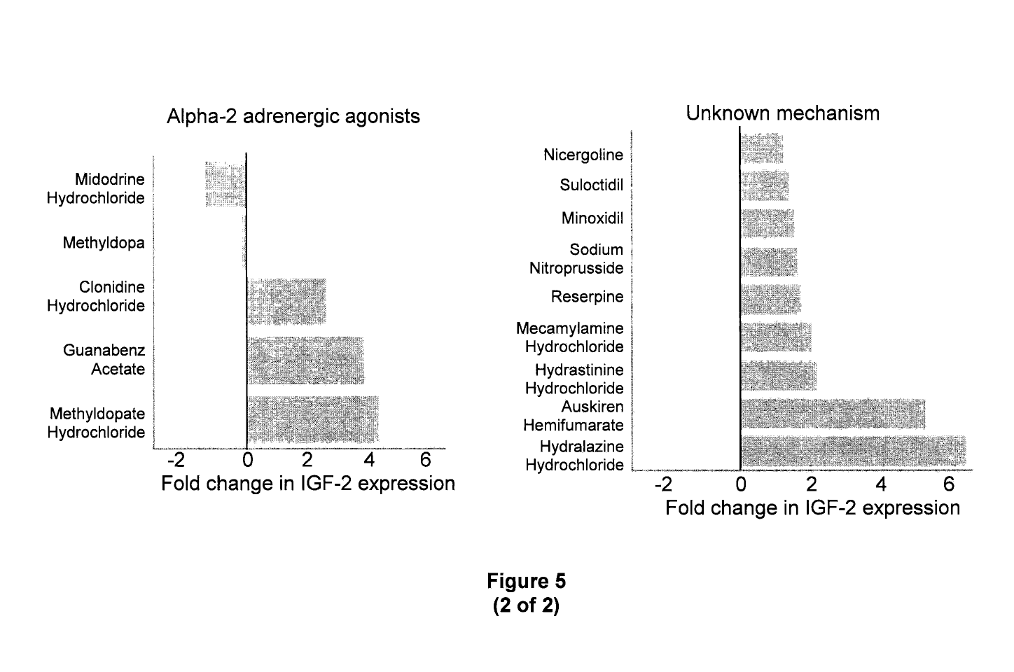

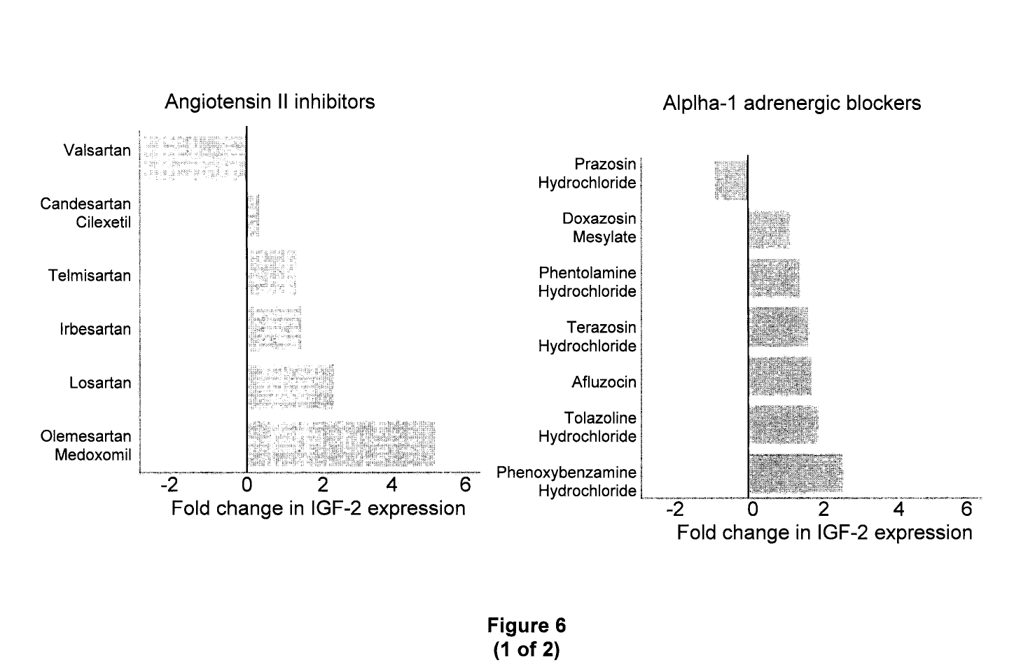

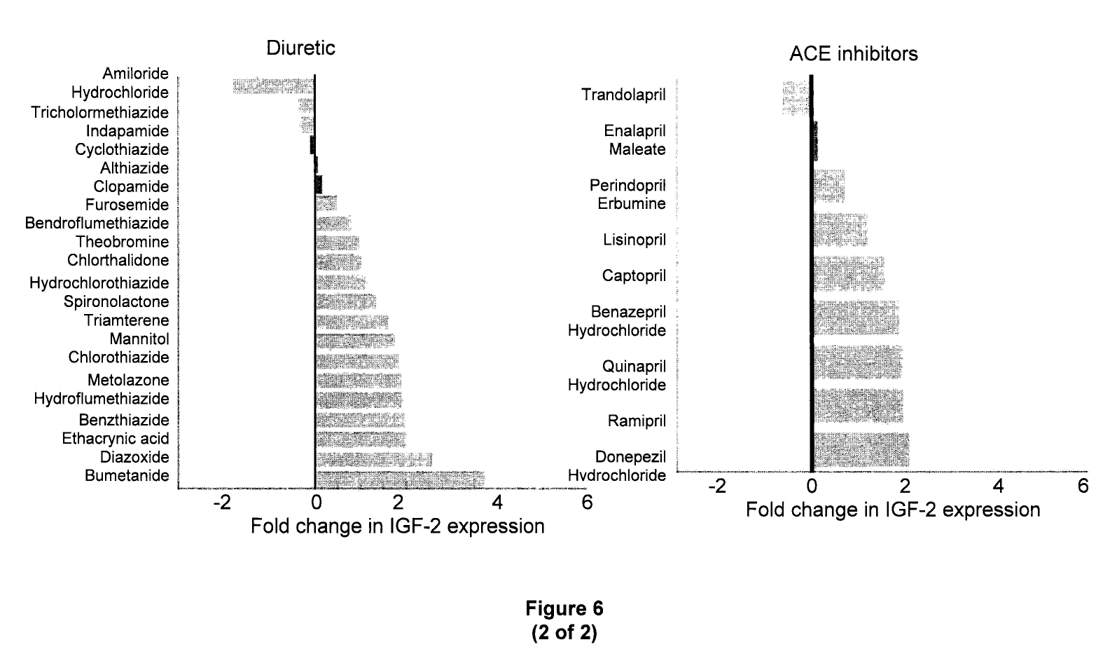

FIGS. 5 and 6 shows the results for the induction of IGF-II expression in a series of antihypertensive agents including calcium channel blockers, beta-adrenergic blockers, alpha-2 adrenergic agonists, a miscellaneous class of antihypertensives, angiotensin II inhibitors, alpha-1 adrenergic blockers, diuretics, and ACE inhibitors. The most active of these includes verapamil, diltiazem HCl, nifedipine, terbutaline hemisulfate, atenolol, timolol maleate, carvedilol, clonidine HCl, guanabenz acetate, methyldopate HCl, aliskiren hemifumarate, hydralazine HCl, olmesartan medoxomil, phenoxybenzamine HCl, bumetanide, and donepezil HCl.

The following table shows the results of selected drugs on IGF-II mRNA and protein expression in primary spinal cord cultures. Of these, the 39 drugs shown were selected for testing in in vitro glutamate toxicity experiments and phenotype shift analysis.

Further results of the library screening identified the following therapeutic agents as having a significant inducing effect on IGF-II expression in primary spinal cord cultures and are useful for treating motor neuron disease. Protein expression was quantitated by ELISA.

| | | Fold Increase in | |
|---|---|---|---|
| | Fold Increase in | IGF-II Protein | |
| | IGF-II mRNA | Expression | |
| Therapeutic Agent | Expression | (pg/ml in media) | Drug Class |
| Eletriptan HBr | 4.3 | | 5-HT agonist, anti-migrane |
| Modafinil | 4.4 ± 4.0 | 1.4 | Analeptic indicated for |
| | | (213 ± 22) | narcolepsy, obstructive sleep |
| | | | apnea, |
| Dicloxacillin sodium | 3.2 | | antibacterial |
| Thiamphenicol | 3.5 | | antibacterial |
| Ceftibuten | 3.9 ± 2.7 | | antibacterial |
| Tacrine HCl | 3.7 | | Anticholinesterase, K channel |
| | | | blocker |
| Fluoxetine | 4.3 ± 2.3 | | antidepressant |
| Citalopram | 3.4 | | antidepressant |
| Fluvoxamine maleate | 4.8 | | antidepressent |
| Amoxapine | 4.1 ± 2.3 | | Antidepressant |
| Atomoxetine HCl | 4.8 | | NE reuptake inhibitor |
| Olmesartan medoxomil | 5.2 | | antihypertensive |
| Guanabenz acetate | 3.8 | | antihypertensive |
| Hydralazine HCl | 6.3 | | antihypertensive |
| Methyldopate HCl | 4.3 | | antihypertensive |
| Diltiazem HCl | 4.3 | | antihypertensive |
| Glyburide | 4.9 | | antihypertensive |
| Flurbiprofen | 2.5 | 1.2 | Anti-inflammatory/analgesic |
| | | (121 ± 27) | |
| Carboprofen | 2.4 | | Anti-inflammatory/analgesic |
| Meloxicam sodium | 4.1 | | Anti-inflammatory/analgesic |
| Diclofenac | 4.0 ± 1.1 | 1.1 | Anti-inflammatory/analgesic |
| | | (112 ± 29) | |
| Levodopa | 3.5 | | Anti-parkinsonian |
| Olanzapine | 4.0 | | Antipsychotic |
| Chlorpromazine | 3.4 | | Antipsychotic |
| Valacyclovir HCl | 4.2 ± 1.8 | 1.5 | Antiviral |
| | | (232 ± 4) | |
| Levocarnitine | 5.7 ± 1.1 | 1.7 | Hepatic carnitine replenisher |
| | | (258 ± 7) | |
| Ropinirole | 4.5 ± 3.0 | | Selective D2-receptor agonist |
| Vardenafil HCl | 6.5 ± 0.2 | 1.6 | PDE5 inhibitor |
| | | (245 ± 27) | |
| Guaifenesin | 2.9 | | Expectorant |
| Omeprazole | 5.9 | | Anti-ulcer |
| Cetirizine HCl | 5.8 | | H1 antihistamine |
| Azelastine HCl | 3.2 | | H1 antihistamine |
| Ramelteon | 3.4 ± 1.3 | 1.5 | Melatonin receptor agonist |
| | | (201 ± 10) | |
| Nicotine ditartrate | 4.8 ± 0.3 | 1.5 | nAChR agonist |
| | | (205 ± 26) | |
| Zolpidem | 6.1 | | Sedative |
| Aspartame | 5.5 ± 0.7 | 1.2 | Sweetener |
| | | (175 ± 1.7) | |
| Thiamine pyrophosphate | 4.1 ± 2.1 | 0.9 | Vitamin B1 |
| | | (118 ± 42) | |
| Riboflavin | 4.1 ± 1.9 | 1.5 | Vitamin B2 |
| | | (205 ± 42) | |
| Niacinamide | 5.0 ± 3.6 | 1.1 | Vitamin B3 |
| | | (151 ± 45) | |
| Dexamethasone acetate | 3.8 | | Glucocorticoid |
| Nifedipine | 6.6 ± 6.2 | | L-type calcium channel |
| | | | antagonist with CNS availability |
| Flurbiprofen | 2.5 ± 0.5 | | NSAID |

### Example 3

### Effect of IGF-II Up-Regulating Drugs on Low-Level Progressive Glutamate Excitotoxicity in Primary Spinal Cord Cultures In Vitro

Primary spinal cord cultures from E12 pregnant mice were pre-treated for 72 hours with 1 μM, 2.5 μM, 5 μM, or 10 μM of a selected drug according to prior optimized dosing regimen. Drugs tested were identified during screening of qPCR. Subsequently, experimental cultures were grown in the presence of 0, 1.0 μM, 2.5 μM, or 10 μM drug for 24 hours prior to exposure to two glutamate (20 μM)/PDC (1 μM) or glutamate (50 μM)/PDC (10 μM) treatments 48 hours apart. Control samples (no glutamate/PDC treatment) were cultured for 24 hours in the presence of 10 μM drug administered 3 times to determine toxicity of treatment. All cultures were then fixed and stained with ChAT, Hoechst stain of the nuclei, and Islet1/2 antibody labels for the motorneuron columns. Results were analyzed using an In Cell Analyzer (GE Healthcare). Results were presented as percent of vehicle (DMSO) treated control cells.

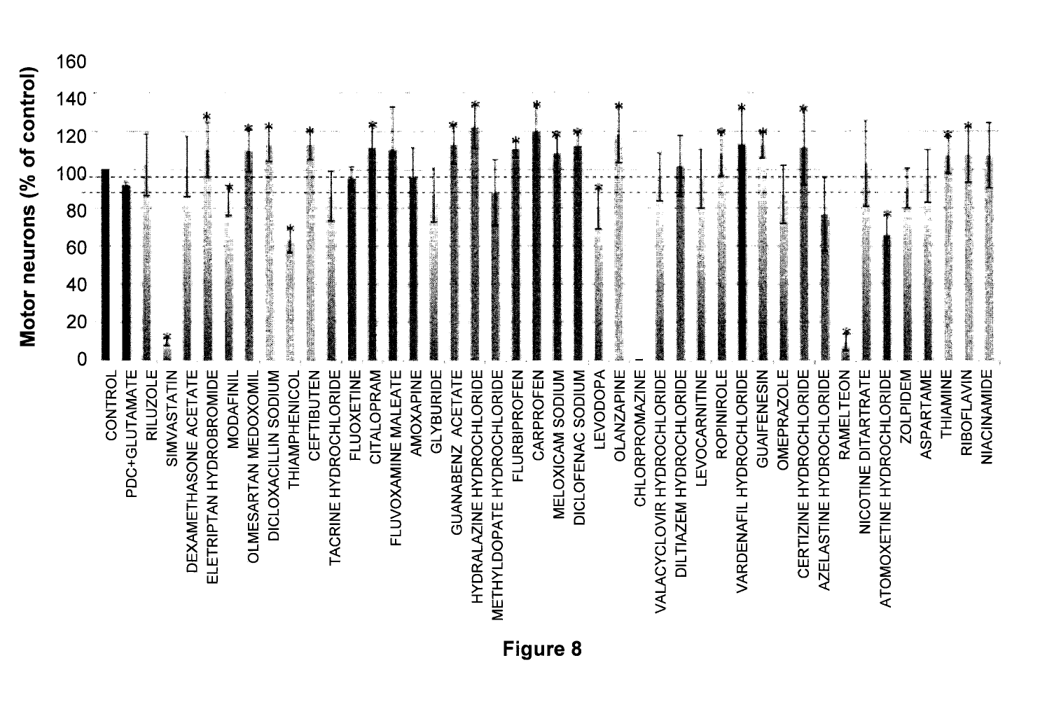

The results, which appear in a bar graph in FIG. 8, demonstrate the percentage of motor neurons as compared to vehicle treated control. Asterisks indicate a resulting number of motor neurons are different from glutamate/PDC treated low toxicity condition. The percentage of motor neurons surviving the glutamate (50 μM)/PDC (10 μM) treatment were about 10-40% of untreated or DMSO vehicle treated control cells. Cells that had been pretreated with drugs (according to above protocol), that up-regulate IGF-II mRNA in the small library screen, were in some cases and to a varying degree protected against excitotoxic insult as evidenced by increased percentage of surviving motor neurons remaining in the cultures. Vardenafil HCl and guanabenz acetate were among the drugs that provided a level of protection.

### Example 4

### Luciferase Reporter Assay (LDDN) to Screen 50,000 Drugs for Inducers of IGF-II Promoter Activation

pGL4.17[luc/Neo] reporter constructs were transfected to contain the P4 IGF-II promoter using the Blue Heron Bio GeneMaker. The mRNA transcript of the IGF-II gene, including the P4 promoter regions, is shown in FIG. 7. After transfection of reporter constructs into SK-N-FI cells+IGF-II using G418 selection pressure for generation of stable cell-line, clonal expansion, IGF-II up-regulating drugs such as glucocorticoids were added, and luminescence was measured. The cell lines were optimized for luciferase intensity, background, and responsiveness. The transfected cells were then used to screen the drug libraries described above

### Effect of IGF-II Up-Regulating Drugs from 1040 Library on P4 Promoter Line

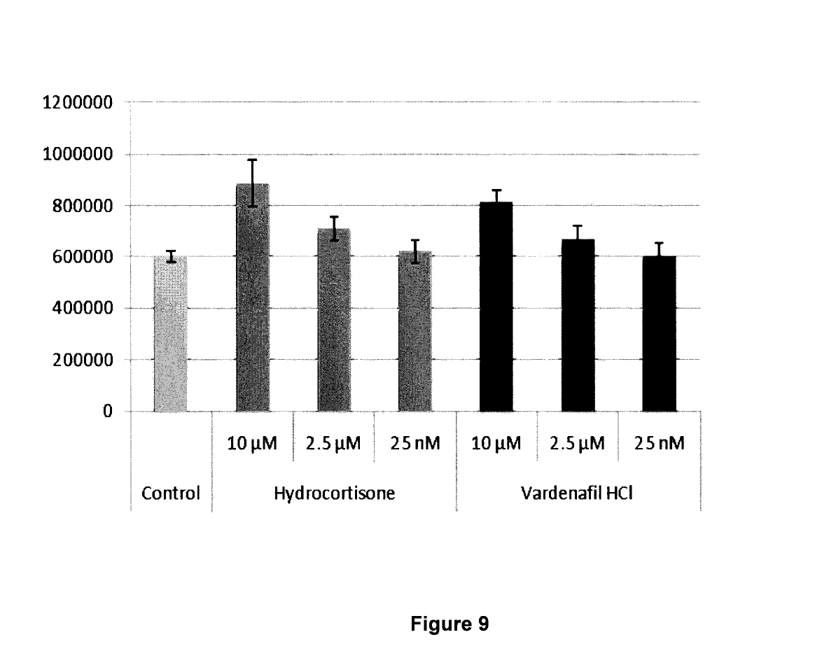

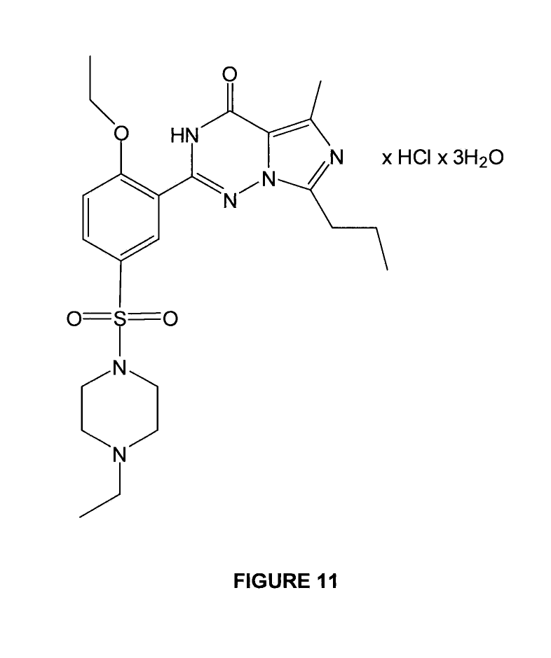

The P4 promoter cell line was cultured as described above. Eight drugs that were positive in the small library screen were tested as described above, and luciferase luminescence was measured. Of the eight tested drugs, four acted on the P4 IGF-II promoter. The best-performing non-glucocorticoid candidate that was tested was Vardenafil HCl. As shown in FIG. 9, the results were roughly equal to those of Hydrocortisone at three concentrations. Vardenafil HCl is a phosphodiesterase type 5 (PDE5) inhibitor with a structure as shown in FIG. 11 and is known to increase cellular cGMP. Vardenafil has been previously FDA approved for treatment of erectile dysfunction and is also clinically used for treating pulmonary hypertension, and is in preclinical studies as a treatment of cystic fibrosis.

The present results indicate that Vardenafil HCl up-regulates IGF-II mRNA 6.5-fold and IGF-II protein 1.6-fold in primary spinal cord cultures from embryonic mice. This results in an activation, as shown in FIG. 9, at a level similar to the known P4 activator hydrocortisone. Further, results indicate that Vardenafil HCl rescues primary motor neurons from low-level progressive glutamate toxicity.

### Example 5

### Luciferase Reporter Assay (LDDN) Using Combined P3 and P4 IGF-II Promoter Construct

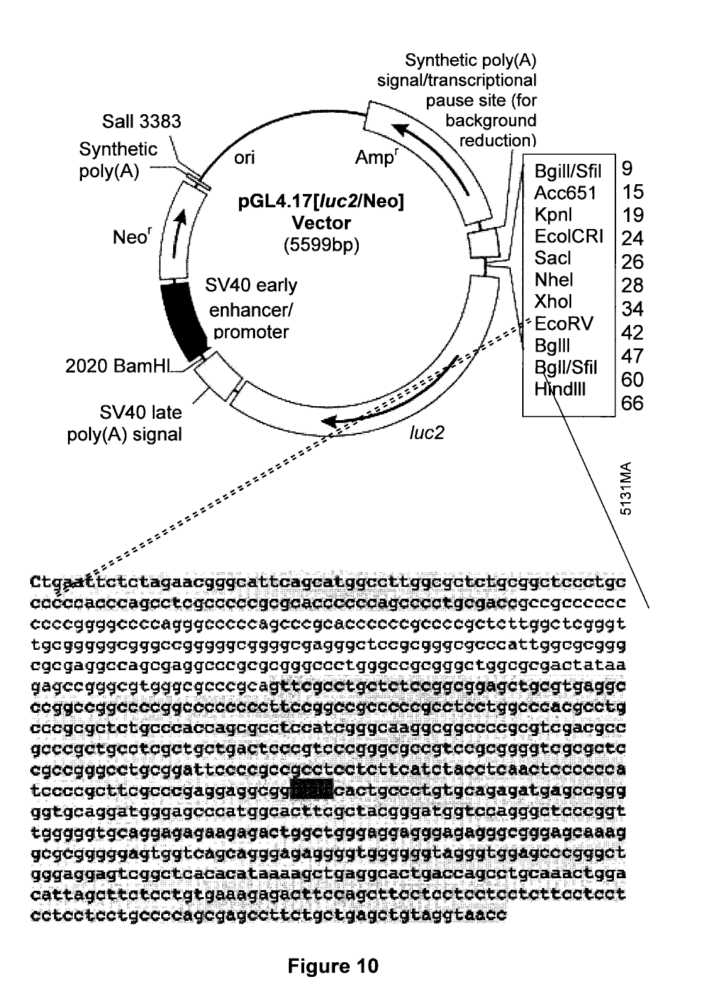

In order to generate a more robust response of candidate drugs, synthesis of a combined P3 and P4 construct may be produced (Blue Heron Biotechnology/OriGene Biotechnology). Since P3 is GC rich and, therefore, contains CpG islands which are a target for methylation in mammalian cell lines, the P3P4 lines are tested for methylation status prior to each screen. The P3P4 construct (SEQ ID NO:14) is transfected in to the pGL4[luc2/Neo] vector at the EcoRV site after the latter undergoes treatment with a restriction endonuclease. A representation of the vector structure is shown in FIG. 10.

After transfection of P3P4 reporter constructs into SK-N-FI cells using G418 selection pressure for generation of stable cell-line, clonal expansion, IGF-II up-regulating drugs such as glucocorticoids are added, and luminescence is measured.

### Example 6

### Vardenafil HCl In Vivo Experiments

Any drug identified as increasing IGF-II expression including, for example, Vardenafil HCl, is tested for in vivo efficacy. The drug or vehicle control are administered to wild type or SOD1 transgenic rodents; optimally fifteen per group. After two weeks, fresh dissection of the spinal cord of approximately three subjects is conducted to determine whether there is upregulation of IGF-II mRNA and/or protein levels. Bi-weekly monitoring of the subjects to test grip-strength, rotarod, traversal beam, and gait is performed. The symptomatic SOD1 rodents are treated with Vardenafil HCl or vehicle in WT and SOD1 rodents. After the endpoint (when a righting reflex test reveals severe immobility), post-mortem analysis is conducted.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for treating a motor neuron disease in a human patient comprising administering to said patient a therapeutically effective amount of an agent that up-regulates IGF-II gene expression.

2. The method of claim 1, wherein the therapeutic agent up-regulates IGF-II by at least 2-fold in said patient.

3. The method of claim 1 or 2, wherein the agent is selected from the group consisting of eletriptan hydrobromide, modafinil, dicloxacillin sodium, thiamphenicol, ceftibuten, tacrine HCl, fluoxetine, citalopram, fluvoxamine maleate, amoxapine, atomoxetine HCl, olmesartan medoxomil, guanabenz acetate, hydralazine HCl, methyldopate HCl, diltiazem HCl, glyburide, flurbiprofen, carprofen, meloxicam sodium, diclofenac sodium, levodopa, olanzapine, chlorpromazine, valacyclovir HCl, levocarnitine, ropinirole, vardenafil HCl, guaifenesin, omeprazole, cetirizine HCl, azelastine hydrochloride, ramelteon, nicotine ditartrate, zolpidem, aspartame, thiamine, riboflavin, niacinamide, sildenafil HCl, tadalafil HCl and dexamethasone acetate.

4. The method of claim 3, wherein the agent is vardenafil HCl.

5. The method of claim 4, wherein the therapeutically effective amount is from 1 mg to 50 mg per day.

6. The method of claim 3, wherein the agent is guanabenz acetate.

7. The method of claim 6, wherein the therapeutically effective amount is from 1 mg to 10 mg per day.

8. The method of claim 1, wherein the motor neuron disease is selected from the group consisting of: amyotrophic lateral sclerosis (ALS), progressive bulbar palsy, spinobulbar muscular atrophy, pseudobulbar palsy, primary lateral sclerosis, progressive muscular atrophy (PMA), spinal muscular atrophy, and post-polio syndrome.

9. (canceled)

10. The method of claim 1, wherein the agent is administered under a dosing regimen that results in at least a 2-fold increase of IGF-II mRNA in the motor neurons of said patient.

11. A method of screening a drug for activity against a motor neuron disease comprising the steps of treatment of cells with the drug, measuring the level of IGF-II, comparing the level of IGF-II in the treated cells with a control, and determining that, if the level of IGF-II has increased between 2-fold and 10-fold, the drug has activity against a motor neuron disease.

12. The method of claim 11, wherein the level of IGF-II has increased between 3-fold and 8-fold.

13. The method of claim 11, wherein the step of level of IGF-II is determined by measuring the level of IGF-II mRNA.

14. A method for treating a motor neuron disease in a human patient comprising administering to said patient a therapeutically effective amount of an agent that up-regulates guanine deaminase gene expression.

15. The method of claim 14, wherein the therapeutic agent up-regulates guanine deaminase by at least 2-fold in said patient.

16. The method of claim 14, wherein the agent is selected from the group consisting of eletriptan hydrobromide, modafinil, dicloxacillin sodium, thiamphenicol, ceftibuten, tacrine HCl, fluoxetine, citalopram, fluvoxamine maleate, amoxapine, atomoxetine HCl, olmesartan medoxomil, guanabenz acetate, hydralazine HCl, methyldopate HCl, diltiazem HCl, glyburide, flurbiprofen, carprofen, meloxicam sodium, diclofenac sodium, levodopa, olanzapine, chlorpromazine, valacyclovir HCl, levocarnitine, ropinirole, vardenafil HCl, guaifenesin, omeprazole, cetirizine HCl, azelastine hydrochloride, ramelteon, nicotine ditartrate, zolpidem, aspartame, thiamine, riboflavin, niacinamide, sildenafil HCl, tadalafil HCl, and dexamethasone acetate.

17. (canceled)

18. The method of claim 17, wherein the therapeutically effective amount is from 1 mg to 50 mg per day.

19. (canceled)

20. The method of claim 19, wherein the therapeutically effective amount is from 1 mg to 10 mg per day.

21. The method of claim 14, wherein the motor neuron disease is selected from the group consisting of: amyotrophic lateral sclerosis (ALS), progressive bulbar palsy, spinobulbar muscular atrophy, pseudobulbar palsy, primary lateral sclerosis, progressive muscular atrophy (PMA), spinal muscular atrophy, and post-polio syndrome.

22. (canceled)

23. The method of claim 14, wherein the agent is administered under a dosing regimen that results in at least a 2-fold increase of IGF-II mRNA in the motor neurons of said patient.
